(19) United States
(12) Patent Application Publication
(10) Pub. No.: US 2011/0274618 A1
(43) Pub. Date: Nov. 10, 2011

(54) DIAGNOSTIC AND/OR THERAPEUTIC AGENT, METHOD FOR THE MANUFACTURE THEREOF AND USE THEREOF

(75) Inventors: Klaus Braun, Sandhausen (DE); Michael Bock, Heidelberg (DE); Ruediger Pipkorn, Heidelberg (DE); Waldemar Waldeck, Laudenbach (DE); Manfred Wiessler, Flomersheim (DE); Bernd Didinger, Koblenz (DE); Juergen Debus, Heidelberg (DE); Volker Ehemann, Sandhausen (DE); Lothar Dunsch, Dresden (DE)

(73) Assignee: Leibniz-Institut Fuer Festkoerper-Und Werkstoffforschung Dresden E.V., Dresden (DE)

(21) Appl. No.: 13/128,560

(22) PCT Filed: Nov. 10, 2009
(86) PCT No.: PCT/EP2009/064912

§ 371 Date: Jul. 27, 2011

(30) Foreign Application Priority Data
Nov. 11, 2008 (DE) 10 2008 043 654.2

(51) Int. Cl. A61K 51/08; C12N 9/96; C07K 16/00; C12Q 1/02; A61P 35/00; A61K 49/04; C07K 1/107; A61K 49/14; A61K 38/02; C07K 2/00; C12Q 1/68

(52) U.S. Cl. 424/1.69; 530/300; 530/322; 435/188; 530/391.1; 435/29; 435/6.1; 424/9.4; 530/345; 424/9.34; 514/1.1

(57) ABSTRACT

The invention relates to the fields of materials sciences and medicine and relates to an agent, which can be used, for example, as a contrast medium for the localization of cancer cells. The object of the present invention is to disclose an agent which sensitively and selectively recognizes the site and the type of the molecules or cells to be examined. The object is attained through an agent composed at least of bio-shuttle molecules to which endohedral fullerenes are coupled by way of peptide-based molecules, wherein the endohedral fullerenes are hydrophobic and correspond to the formula A₃₋ₓMₓZ@C₂ₙ in which x=0 to 3 and n≧34, A means rare earths and/or transuranic elements, M means metals, Z means non-metals and C means carbon. The object is further attained through a method in which hydrophobic endohedral fullerenes are coupled with bio-shuttle molecules by way of an irreversible Diels-Alder reaction with an inverse electron demand (DARᵢₙᵥ).

The invention relates to the fields of materials sciences and medicine and relates to a diagnostic and/or therapeutic agent, which can be used, for example, as a contrast medium for the diagnostic and therapeutic application for the localization of cancer cells, for establishing the type of cancer cells or for the destruction of cancer cells, as well as a method for the production thereof.

Contrast media can be administered in medical imaging methods, for example, x-ray imaging, nuclear resonance imaging or ultrasound imaging, in order to enhance or reduce the image contrast in images of subjects, in general of a human or animal body. The contrast increased thereby makes it possible to observe or to identify different organs, types of tissue and parts of the body more clearly or to examine the function of the organs. In the case of x-ray imaging, contrast media modify the x-ray absorption characteristics of the regions of the body in which they are disbursed. Nuclear resonance contrast media in general modify the density or the characteristic relaxation times of the nuclei, in general water protons, from the resonance signals of which the images are produced. Contrast media for positron emission tomography (PET) act as a source of a detectable radiation. Magnetometric contrast media act by creating perturbations of the magnetic field in the regions of the body in which they are disbursed. These perturbations can be detected, for example, by means of SQUID magnetometers. Ultrasound contrast media modify the sound velocity or the density in the parts of the body in which they are disbursed.

The improvement of contrast media with respect to contrast intensification, biodistribution, stability, opacity, relaxivity, tolerance, etc., however, is a central topic of scientific studies.

According to DE 693 28 550 T2, the use of fullerene derivatives in diagnostic and/or therapeutic agents is known. The fullerene derivatives are thereby used as contrast enhancement agents and as carriers for signal-forming units that can be attached to biomolecules.

An optoacoustic contrast medium and a method for the use thereof are furthermore known (DE 698 31 755 T2). An agent is thereby administered to the patient, which agent contains a stabilizing material, a photoactive agent and a gas. By means of ultrasound the stabilizing material is ruptured and the photoactive agent is released. Subsequently, light energy is used to activate the photoactive agent.

From DE 600 14 124 T2 a fullerene-specific antibody is known, which can specifically bind fullerenes and fullerene nanotubes. Furthermore, a method is known for determining the fullerene concentration in the serum of a patient.

According to DE 103 01 722 A1 a method is known for producing endohedral fullerenes. Accordingly, the endohedral fullerenes are guided through an atmosphere in an arc reactor by atomizing graphite electrodes, which atmosphere contains a reactive gas component composed of at least two elements in an inert gas or an inert gas mixture.

Progress in the precision of diagnostics has not been able to keep pace with progress made in therapy. The solutions of the prior art have in common that the currently known contrast media do not adequately meet the demands of modern diagnostics and therapy with respect to contrast enhancement, biodistribution, stability, opacity, relaxivity, tolerance, etc.

The solution according to the invention closes this gap. The object of the present invention is to disclose a diagnostic and/or therapeutic agent, which sensitively and selectively recognizes the site and the type of the molecules or cells to be examined and can be used just as selectively therapeutically, as well as an effective and selective method for the production thereof and the use thereof for different imaging methods.

The object is attained through the invention disclosed in the claims. Advantageous embodiments are the subject matter of the subordinate claims.

The diagnostic and/or therapeutic agent according to the invention with at least one imaging component on the transcription level is composed at least of bio-shuttle molecules to which endohedral fullerenes are coupled by way of peptide-based molecules, wherein the endohedral fullerenes are hydrophobic and correspond to the formula

A₃₋ₓMₓZ@C₂ₙ

in which x=0 to 3 and n≧34,
and in which A means rare earths and/or transuranic elements, M means metals, Z means non-metals and C means carbon.

Advantageously, the hydrophobic endohedral fullerenes contain nitride clusters of metals, rare earths and/or transuranic elements individually or in a mixture.

Likewise advantageously, the metals, rare earths and/or transuranic elements have a magnetic moment.

Furthermore advantageously, as A, rare earths, gadolinium, holmium, dysprosium, lanthanum, ytterbium and/or terbium are present.

Also advantageously, as A, transuranic elements, neptunium, actinium, uranium and/or plutonium are present.

It is also advantageous if as M, metals, scandium, yttrium, calcium, strontium, barium are present.

It is furthermore advantageous if as Z, nonmetals, N, P, S, B, O and/or compounds thereof and/or the isotopes thereof are present.

It is also advantageous if Z as an isotope is present as ¹⁷O, ¹⁸O, ³⁴S, ³⁵S, ³²P, ³³P.

It is likewise advantageous if the bio-shuttle molecules are composed of a transport module, an address module and a cargo.

It is also advantageous if amphiphilic molecules, such as homeobox-type protein fragments (HOX) are present as a transport module, wherein yet more advantageously P^{AnT} (Antennapedia insects), PTD^{HIV-1/TAT} (viral origin), TP^{IAOP/Eco} (bacterial origin, e.g., Escherichia coli), TP^{human} and/or TP^{variable} are present as HOX protein fragments.

It is also advantageous if peptide nucleic acid sequences (PNA), nucleic acids and/or peptide fragments are present as intracellular address molecules.

It is likewise advantageous if aberrant gene expression of cell cycle control genes, apoptosis inhibitor genes, matrix metal proteinases (MMPs), RNA such as e.g.: fusion mRNA of fusion genes, RNA, expressed in stem cells, and/or antibody fragments, substrates for enzyme-specific cleavage are present.

It is furthermore advantageous if peptide sequences and/or antisense peptide nucleic acid (AS)-PNA are present as an address module.

It is also advantageous if a peptide sequence protease-cleavable by the enzyme cathepsin B or another specific protease-cleavable peptide sequence is present as an address module.

It is also advantageous if the cargo is composed of endohedral fullerenes.

In the method according to the invention for producing a diagnostic and/or therapeutic agent, hydrophobic endohedral fullerenes are coupled with bio-shuttle molecules by way of an irreversible Diels-Alder reaction that has an inverse electron demand (DARᵢₙᵥ).

Advantageously, the coupling is realized by covalent bonds.

Likewise advantageously, the DAR with inverse electron demand makes a back reaction impossible, so that no chemical equilibrium is set between the fullerenes and the derivatives thereof and the bio-shuttle molecules.

Furthermore advantageously, the central part of the fullerene bio-shuttle is produced via a dienophile such as boc-K(TCT)-OH and a tetrazine at room temperature in the solid phase synthesis.

According to the invention, the diagnostic and/or therapeutic agent is used as a component for the molecular imaging and/or for the selective and/or complete destruction of cells, the contents and/or structures thereof e.g. nuclei.

Advantageously, it is used as a component for molecular imaging for x-ray, MRT, SPECT, PET examinations or and/or for therapies such as, e.g., BNCT therapy approach (boron neutron capture therapy) or with modern chemotherapies (reformulated and/or patient-specific therapy approaches).

Furthermore advantageously, the diagnostic and/or therapeutic agent is used as an intracellular/intravital contrast medium.

Also advantageously, the diagnostic and simultaneously therapeutic agent is used using a magnetic field for monitoring the course of therapy without radiation exposure for the patient.

With the solution according to the invention, it is possible for the first time to provide a diagnostic and/or therapeutic agent, which acts in an intracellular/intravital manner, acts very selectively, realizes a high-contrast molecular imaging (molecular imaging=imaging of metabolic processes on the transcription level) and in certain cases can also act therapeutically. Due to a lack of sensitivity, it has hitherto been impossible to image intracellular processes selectively via MR tomography. A differentiation of e.g., tumor tissue and surrounding healthy tissue, and a distinction between necrotic regions have not been possible until now. For this reason the imaging of micrometastases has therefore not been possible with the previous methods so far, either.

This imaging method provided by the solution according to the invention now also renders possible through the use of the diagnostic and/or therapeutic agent according to the invention (as an intracellular contrast medium) the tomographic representation of organs on a metabolic level also.

Through the method according to the invention it is possible for the first time to bind hydrophobic endohedral fullerenes to bio-shuttle molecules so that biomolecules and address molecules are available that are present bound irreversibly to the fullerenes via covalent bonds. Thus with the introduction of the agent according to the invention, for example, into a human body or an animal body, the agent is locked into the respectively desired cell (target cell) and there, after hybridization with the respectively aberrantly expressed target mRNA (in this case CTBS mRNA=overexpressed cathepsin B) via the hydrophobic endohedral fullerenes, can be localized in an imaging manner.

Structure and composition of the hydrophobic endohedral fullerenes can be adapted to the respective purpose (molecular target).

The special advantage of the hydrophobic endohedral fullerenes according to the invention is that in the interior of the carbon cage they can contain rare earths and/or transuranic elements, metals and nonmetals, which overall are suitable for a very good imaging on the molecular level (transcription level). Substances harmful to the human or animal body can definitely be used thereby. Since these are held safely “captured” by the carbon cage and there is thereby no direct contact with cells and their active components, such as enzymes of human and animal bodies.

While the previously commercially available contrast media have been substances that cannot penetrate the cell membrane, the following applications of the solution according to the invention including the use as a contrast medium in various diagnostic procedures as well as a therapeutic agent are new and advantageous. The agent according to the invention thereby penetrates into the respectively desired cells, for example, cancer cells, in a targeted manner and accumulates there (increased local concentration). Special peptide sequences thereby form a substrate for cell-imminent mechanisms which render possible an active transport, e.g., into the nucleus (nuclear localization sequence=NLS; or mitochondrial localization sequence=MLS).

By means of the imaging methods described here, the concentration of the agent according to the invention can be determined tomographically in several cells (micrometastases) individually or for the first time in individual cells (e.g., tumor stem cells); the cells are thus localized very precisely; the gene expression profile (transcript) of the respective cell (malignant or benign tumor) can be detected in this manner and permits a clear selection of tumor tissue and surrounding normal tissue.

To use the agent according to the invention for imaging methods, it is particularly advantageous if individual or all substances in the hydrophobic endohedral fullerenes have a magnetic moment.

Depending on the respective compositions of the hydrophobic endohedral fullerenes, individual or all constituents can be activated by special methods at the respectively desired sites and thus, for example, one cell or the cell content or the cell nucleus can be irreparably destroyed.

Other detections via hybridizations depending on the issue are also possible, for example for the first time imaging in the MRT of stem cells, the migration of which is also possible in the molecular MR imaging based on the typical gene expression profile, or the imaging (monitoring) of the migration of micrometastases can be carried out!

The invention is explained in more detail below based on exemplary embodiments.

They show:

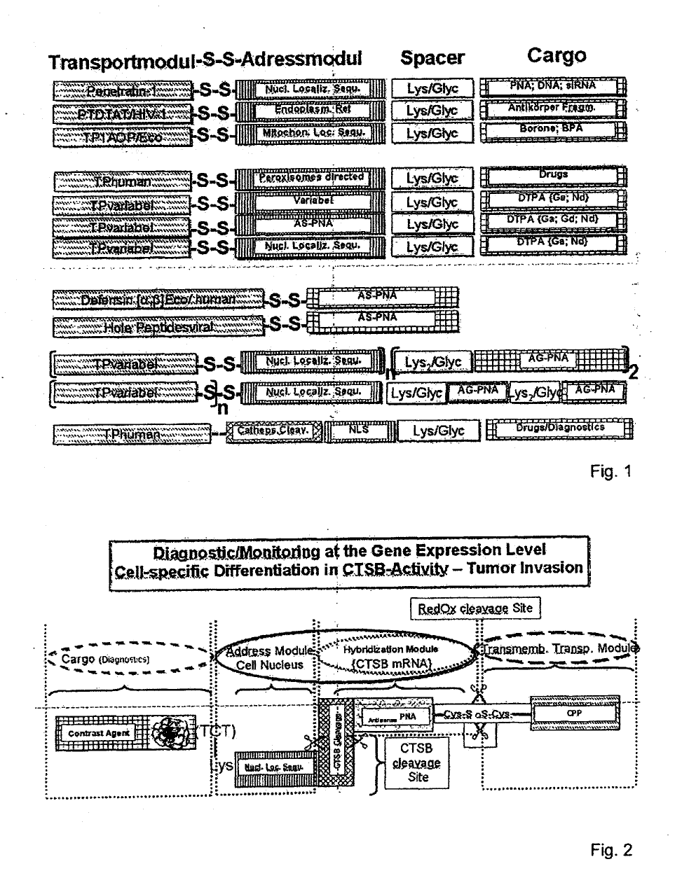

FIG. 1 The diagrammatic composition of transport module, address module and cargo of the agent according to the invention; transport module (1^{st} module=left column), address module (2^{nd} module) and cargo (3^{rd} and 4^{th} module) for different molecule variants.

FIG. 2 The diagrammatic structure of a Gd-cluster@bio-shuttle according to the invention with the transport module on the right, with the address module in the center and the cargo on the left.

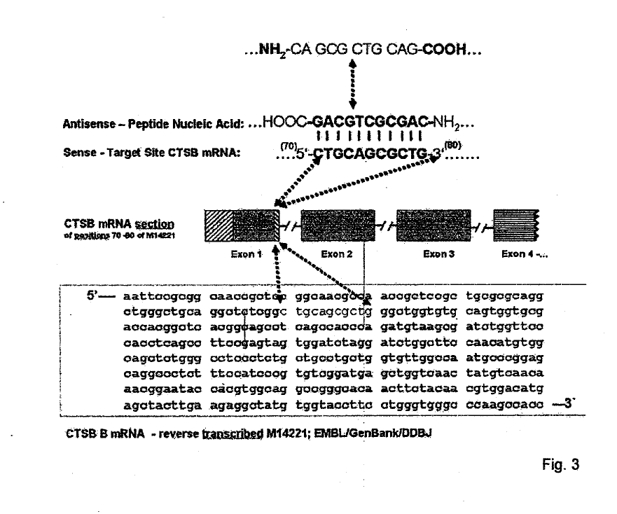

FIG. 3 The molecular action site of the transported PNA (at the top) on the CTSB mRNA (3′ end of exon 1); AC number can be found in the lower part of the figure.

### EXAMPLE 1

In an arc reactor, graphite electrodes modified with gadolinium metal and scandium metal are removed in a gas mixture, which contains a reactive gas component, with pulsed direct current with a current strength between 75 A and 150 A. The graphite electrodes used have a composition with the ratio graphite:gadolinium and graphite:scandium of respectively 1 mol:0.4 mol. The gas mixture is composed of He and NH₃, wherein the NH₃ is the reactive component. The proportions in the gas mixture are 20 kPa He and 2 kPa NH₃.

In the implementation of this method, endohedral mixed gadolinium-scandium nitride cluster fullerenes are produced with a yield between 5 and 35%.

The synthesis of the acid chloride of the fullerene is carried out according to a specification by J. Arrowsmith et al. (J. Med. Chem. 2002; 45: 5458-70). To this end, first 2 mmol of the acid is reflux boiled with 10 ml thionyl chloride until the acid is completely dissolved. The excess of thionyl chloride is separated and the precipitate obtained is dried over NaOH in the desiccator overnight.

In the second step the synthesis of the boc-propyldiamine-tetrazine-diene is carried out. To this end 2 mmol of the acid chloride is suspended in 20 ml abs. dichloromethane and then a mixture of 2 mmol N-boc-1,3-diaminopropane and 2 mmol triethylamine is slowly added to 10 ml of the same solvent at 0-5° C. The strongly colored solution obtained is held for 4 hours at room temperature and subsequently the organic phase is washed with water, 1 N-HCL and again with water. After the drying with Na₂SO₄, the filtration and the evaporation of the residual solvent, the residue is cleaned chromatographically (on silica gel) with chloroform/ethanol 9:1 as solvent and then recrystallized from acetone. The yield is 70%, but always also depends on the quality of the respective carboxylic acid. The mass spectrometry (ESI) showed: positive ions m/e 337.1.

Both of the following compounds produce the fullerene-_{(aminobound)}-tetrazoline-diene according to the following specification:

0.5 mmol of the mono-substituted tetrazine-amine and 0.5 mmol of 4-methyl-5-oxo-2,3,4,6,8-pentazabicyclo[4.3.0]nona-2,7,9-triene-9-carboxylic acid chloride is dissolved in 5 ml chloroform and 5 ml triethylamine (v/v=1:1) at 0-5° C. After 4 h at room temperature, the solution is washed with water, 1N hydrochloric acid and again with water. The reaction runs uninterrupted and after 6 h yields the product. After the drying and evaporation of the residual solvent, the remaining residue is cleaned chromatographically with chloroform/ethanol 9.5/0.5 as a solvent (over silica gel). The mass spectrometry (ESI) showed: positive ions m/e 536.3 (+Na).

The target cell specificity is based on a specific gene expression profile in target cells. The target cell-specific mRNAs (in this case CTSB mRNA=cathepsin B mRNA) are hybridized with complementary PNAs (peptide nucleic acids), in the cytoplasma. Since PNA/mRNA hybrids do not represent a substrate for RNAse H, these hybrids remain trapped together with the cargo (Gd—Sc-cluster@) in the cytoplasm of the target cells. The first step of the specificity of Gd—Sc-cluster@-bio-shuttle is based on this. The second step is based on the enzymatic cleavage of the PNA on the CTSB interface (CTSB protein=enzyme) which is located between the PNA and the cargo (Gd—Sc-cluster@). After cleavage, the address sequence for the nucleus (nuclear localization sequence=NLS) is freely accessible for molecules that cause the active transport of the cargo (Gd—Sc-cluster@) into the nucleus. The stability of the PNAIRNA hydrides under physiological conditions leads to a passive enrichment of the cargo (Gd—Sc-cluster@) in the cytoplasm; the active transport of Gd—Sc-cluster@ into the nucleus of CTSB of active cells (malignant cells=invasive cells) leads to an active enrichment of the Gd—Sc-cluster@ in the nucleus thereof and gives an MR signal in contrast to normal cells, which eliminate the Gd—Sc-cluster@ again due to a lack of hybridization possibilities. The increased sensitivity compared to known MR contrast media (e.g., Gd chelates or iron oxide particles) is realized through the special properties of the Gd—Sc-cluster@.

### EXAMPLE 2

In an arc reactor a graphite electrode modified with gadolinium metal is burned off in a gas mixture that contains a reactive gas component with pulsed direct current with a current strength between 75 A and 150 A. The graphite electrodes used have a composition with the ratio graphite:gadolinium of 1 mol:0.4 mol. The gas mixture is composed of He and NH₃, wherein the NH₃ is the reactive component. The proportions in the gas mixture are 200 mbar He and 20 mbar NH₃.

In the implementation of this method, endohedral gadolinium nitride cluster fullerenes are produced with a yield between 5 and 10%.

The synthesis of the acid chloride of the fullerene is carried out according to a specification by J. Arrowsmith et al. (J. Med. Chem. 2002; 45: 5458-70). To this end, first 2 mmol of the acid is reflux boiled with 10 ml thionyl chloride until the acid is completely dissolved. The excess of thionyl chloride is separated and the precipitate obtained is dried over NaOH in the desiccator overnight.

In the second step, the synthesis of the boc-propyldiamine-tetrazine-diene is carried out. To this end 2 mmol of the acid chloride is suspended in 20 ml abs. dichloromethane and then a mixture of 2 mmol N-boc-1,3-diaminopropane and 2 mmol triethylamine is slowly added to 10 ml of the same solvent at 0-5° C. The strongly colored solution obtained is held for 4 hours at room temperature and subsequently the organic phase is washed with water, 1 N-HCL and again with water. After the drying with Na₂SO₄, the filtration and the evaporation of the residual solvent, the residue is cleaned chromatographically (on silica gel) with chloroform/ethanol 9:1 as solvent and then recrystallized from acetone. The yield is 70%, but always also depends on the quality of the respective carboxylic acid. The mass spectrometry (ESI) showed: positive ions m/e 337.1.

Both of the compounds yield the fullerene-_{(aminobound)}-tetrazoline-diene according to the following specification:

0.5 mmol of the mono-substituted tetrazine-amine and 0.5 mmol of 4-methyl-5-oxo-2,3,4,6,8-pentazabicyclo[4.3.0]nona-2,7,9-triene-9-carboxylic acid chloride is dissolved in 5 ml chloroform and 5 ml triethylamine (v/v=1:1) at 0-5° C. After 4 h at room temperature the solution is washed with water, 1N hydrochloric acid and again with water. The reaction runs uninterrupted and after 6 h yields the product. After the drying and evaporation of the residual solvent, the remaining residue is cleaned chromatographically with chloroform/ethanol 9.5/0.5 as solvent (over silica gel). The mass spectrometry (ESI) showed: positive ions m/e 536.3 (+Na).

A solution of 0.1 mmol Gd-cluster@ in 35 ml toluene is stirred overnight. The undissolved fractions are filtered off 28 mg propylamine-dihydrotetrazine is added to the solution and stirred for 48 hours at room temperature. The solution is then held again for 24 hours at room temperature and subsequently held over 5 hours at 50° C., filtered and evaporated. Subsequently the oxidation to tetrazine (magenta) takes place.

With the Gd-cluster@bio-shuttle thus produced, the proliferation behavior of tumor cells can be shown in MR imaging using the example of over-expressed c-myc mRNA. Particularly as the T/2 of c-myc mRNA is about 20 min and a hybridization leads to the inhibition of the decomposition thereof, which with further transcription of c-myc RNA leads to an enrichment in the cytoplasma of hybrid c-myc mRNA/PNA. In normal cells, c-myc m-RNA is practically undetectable.

### EXAMPLE 3

In an arc reactor graphite electrodes are burned off in a gas mixture that contains a reactive gas component with a pulsed direct current with a current strength of 175 A. The gas mixture is composed of He and CH₄, wherein the CH₄ is the reactive component. The proportions in the gas mixture are 200 mbar He and 10 mbar CH₄.

In the implementation of this method, CH₂@C₇₀ is produced as the main component of the endohedral fullerenes, wherein C₆₀ and C₇₀ represent the main proportion of the total fullerene content.

The chemical binding of the bio-shuttle module to the endohedral fullerene containing sulfur is carried out as described in example 1.

### EXAMPLE 4

In an arc reactor graphite electrodes modified with lutetium metal and guanidinium rhodanide are burned off with pulsed direct current with a current strength between 75 A and 150 A. The graphite electrodes used have a composition with the ratio graphite:lutetium 1 mol:0.4 mol and guanidinium rhodanide:lutetium 0.5 mol:1 mol. The pressure of the He cooling gas is 200 mbar He.

In the implementation of this method endohedral Lu₂S—C₈₂-fullerenes with a yield between 2 and 5% are produced.

The chemical binding of the bio-shuttle module to the endohedral fullerene containing sulfur is carried out as given in example 2.

1. Diagnostic and/or therapeutic agent with at least with at least one imaging component on the transcription level, composed at least of bio-shuttle molecules to which endohedral fullerenes are coupled by way of peptide-based molecules, wherein the endohedral fullerenes are hydrophobic and correspond to the formula
A₃₋ₓMₓZ@C₂ₙ
in which x=0 to 3 and n≧34,
and in which A means rare earths and/or transuranic elements, M means metals, Z means non-metals and C means carbon.

2. Diagnostic and/or therapeutic agent according to claim 1, characterized in that the hydrophobic endohedral fullerenes contain nitride clusters of metals, rare earths and/or transuranic elements individually or in a mixture.

3. Diagnostic and/or therapeutic agent according to claim 1, characterized in that the metals, rare earths and/or transuranic elements have a magnetic moment.

4. Diagnostic and/or therapeutic agent according to claim 1, characterized in that as A, rare earths, gadolinium, holmium, dysprosium, lanthanum, ytterbium and/or terbium are present.

5. Diagnostic and/or therapeutic agent according to claim 1, characterized in that as A, transuranic elements, neptunium, actinium, uranium and/or plutonium are present.

6. Diagnostic and/or therapeutic agent according to claim 1, characterized in that as M, metals, scandium, yttrium, calcium, strontium, barium are present.

7. Diagnostic and/or therapeutic agent according to claim 1, characterized in that as Z, nonmetals, N, P, S, B, O and/or compounds thereof and/or the isotopes thereof are present.

8. Diagnostic and/or therapeutic agent according to claim 7, characterized in that Z as an isotope is present as ¹⁷O, ¹⁸O, ³⁴S, ³⁵S, ³²P ³³P.

9. Diagnostic and/or therapeutic agent according to claim 1, characterized in that the bio-shuttle molecules are composed of a transport module, an address module and a cargo.

10. Diagnostic and/or therapeutic agent according to claim 1, characterized in that amphiphilic molecules, such as homeobox-type protein fragments (HOX) are present as a transport module.

11. Diagnostic and/or therapeutic agent according to claim 10, characterized in that P^{AnT} (Antennapedia insects), PTD^{HIV-1/TAT} (viral origin), TP^{IAOP/Eco} (bacterial origin, e.g., Escherichia coli), TP^{human} and/or TP^{variable} are present as HOX protein fragments.

12. Diagnostic and/or therapeutic agent according to claim 1, characterized in that peptide nucleic acid sequences (PNA), nucleic acids and/or peptide fragments are present as intracellular address molecules.

13. Diagnostic and/or therapeutic agent according to claim 12, characterized in that aberrant gene expression of cell cycle control genes, apoptosis inhibitor genes, matrix metal proteinases (MMPs), RNA such as e.g.: fusion mRNA of fusion genes, RNA, expressed in stem cells, and/or antibody fragments, substrates for enzyme-specific cleavage are present.

14. Diagnostic and/or therapeutic agent according to claim 12, characterized in that peptide sequences and/or antisense peptide nucleic acid (AS)-PNA are present as an address module.

15. Diagnostic and/or therapeutic agent according to claim 14, characterized in that a peptide sequence protease-cleavable by the enzyme cathepsin B or another specific protease-cleavable peptide sequence is present as an address module.

16. Diagnostic and/or therapeutic agent according to claim 1, characterized in that the cargo is composed of endohedral fullerenes.

17. Method for producing a diagnostic and/or therapeutic agent according to claim 1, in which hydrophobic endohedral fullerenes are coupled with bio-shuttle molecules by way of an irreversible Diels-Alder reaction that has an inverse electron demand (DARᵢₙᵥ).

18. Method according to claim 17, characterized in that the coupling is realized by covalent bonds.

19. Method according to claim 18, characterized in that the DAR with inverse electron demand makes a back reaction impossible, so that no chemical equilibrium is set between the fullerenes and the derivatives thereof and the bio-shuttle molecules.

20. Method according to claim 17, characterized in that the central part of the fullerene bio-shuttle is produced via a dienophile such as boc-K(TCT)-OH and a tetrazine at room temperature in the solid phase synthesis.

21. Use of the diagnostic and/or therapeutic agent according to claim 1 as a component for the molecular imaging and/or for the selective and/or complete destruction of cells, the contents and/or structures thereof, e.g., nuclei.

22. Use according to claim 21, characterized in that the diagnostic and/or therapeutic agent is used as a component for molecular imaging for x-ray, MRT, SPECT, PET examinations or and/or for therapies such as, e.g., BNCT therapy approach (boron neutron capture therapy) or with modern chemotherapies (reformulated and/or patient-specific therapy approaches).

23. Use according to claim 21, characterized in that the diagnostic and/or therapeutic agent is used as an intracellular/intravital contrast medium.

24. Use according to claim 23, characterized in that the diagnostic and simultaneously therapeutic agent is used using a magnetic field for monitoring the course of therapy without radiation exposure for the patient.
